(19) United States
(12) Patent Application Publication
(10) Pub. No.: US 2021/0369383 A1
(43) Pub. Date: Dec. 2, 2021

(54) Face Shield Protection Device

(71) Applicants: Jason Pawlowicz, Lyndora, PA (US); John Pawlowicz, III, Lyndora, PA (US)

(72) Inventors: Jason Pawlowicz, Lyndora, PA (US); John Pawlowicz, III, Lyndora, PA (US)

(21) Appl. No.: 17/333,430

(22) Filed: May 28, 2021

Related U.S. Application Data
(60) Provisional application No. 63/111,114, filed on Nov. 9, 2020.
(60) Provisional application No. 63/031,858, filed on May 29, 2020.

(51) Int. Cl. A61B 90/00

(52) U.S. Cl. CPC A61B 90/05; A61B 2090/571

(57) ABSTRACT

A face shield protection device including a shield assembly having a frame defining an opening and a transparent shield removably attached to the frame and substantially covering the opening, and a suspension arm attached to the shield assembly. The transparent shield is attached to a first side of the frame in a first receiving engagement and is attached to an opposite second side of the frame in a second receiving engagement, and one or both of the first receiving engagement and the second receiving engagement locks the transparent shield with a portion of the frame. The face shield protection device may further comprise at least one nozzle extending beneath a bottom surface of the transparent shield, wherein suction provided through the at least one nozzle pulls atmosphere from beneath the transparent shield creating negative pressure beneath the transparent shield.

### CROSS-REFERENCE TO RELATED APPLICATIONS

This application claims priority to Provisional U.S. Patent Application No. 63/031,858 filed May 29, 2020 and Provisional U.S. Patent Application No. 63/111,114 filed Nov. 9, 2020, the disclosures of which are hereby incorporated by reference in their entirety.

### BACKGROUND OF THE INVENTION

### Field of the Invention

The present invention is directed to a face shield protection device, and more specifically, a face shield protection device used to protect a medical practitioner from exposure to aerosol expelled from a patient's nose and/or mouth while performing a medical or dental procedure.

### Description of Related Art

Generally, when a medical professional such as a doctor or dentist must provide care to the face, mouth, head, or throat area of a patient, either the medical practitioner must wear a face shield and/or suction is provided in the general area of the patients head. Both of these solutions can still expose the practitioner and/or the patient to aerosols expelled from the practitioner's and/or the patient's mouth and/or nose, especially when the practitioner is working above the patient such that aerosols may enter and exit the gap at the bottom of a face shield worn by the practitioner.

Thus, there is a need for an improved face shield protection device to mitigate the spread of the Covid-19 virus and other airborne contaminants and viruses during medical and dental procedures, especially when the medical practitioner is working directly over the patient's face, for example, during a dental procedure.

### SUMMARY OF THE INVENTION

The present invention is directed to a face shield protection device comprising a shield assembly comprising a frame defining an opening and a transparent shield removably attached to the frame and substantially covering the opening, and a suspension arm attached to the shield assembly. The transparent shield is attached to a first side of the frame in a first receiving engagement in which a portion of the transparent shield is received within a portion of the frame and is attached to an opposite second side of the frame in a second receiving engagement in which a portion of the transparent shield is received within an opposing second side portion of the frame, and one or both of the first receiving engagement and the second receiving engagement locks the transparent shield with a portion of the frame.

The frame may comprise a proximal beam, a distal beam, a first side beam extending from a first end of the proximal beam to a first end of the distal beam, and a second side beam extending from a second end of the proximal beam to a second end of the distal beam, wherein the proximal beam, the distal beam, the first side beam, and the second side beam define the opening. The proximal beam, the distal beam, the first side beam, and/or second side beam may be tubular and define a passageway.

The transparent shield may have a proximal end, a distal end, a first side, and a second side which correspond to the proximal beam, the distal beam, the first side beam, and the second side beam, respectively, of the frame. The transparent shield may be a flexible sheet and has a shape that corresponds to the shape of the opening defined by the frame and a top surface of the transparent shield may have a convex curvature in the transverse direction and/or a convex curvature in the longitudinal direction.

The first side beam and the second side beam may be tubular, each defining a passageway, and at least one tab may extend from the first side of the transparent shield and at least one tab may extend from the second side of the transparent shield. The at least one tab on the first side of the transparent shield may be received by a groove in the first side beam of the frame in the first receiving engagement, and the at least one tab on the second side of the transparent shield may be received in an opening through a sidewall of the second side beam of the frame in the second receiving engagement. The at least one tab on the first side of the transparent shield may be locked within the first side beam frame and/or the at least one tab on the second side of the transparent shield may be locked within the second side beam of the frame.

A shaft may be contained within the passageway of the second side beam and at least one engagement member may extend from the shaft. When the at least one tab on the second side of the transparent shield passes into the opening in the second side beam, the engagement member may be configured to, upon rotation of the shaft, engage the at least one tab to lock the at least one tab within the passageway of the second side member. The engagement member may be a protrusion extending from the shaft through the opening, and the at least one tab may be locked between the protrusion and a side of the opening in the second side member. The transparent shield may comprise two tabs extending from opposite ends of the second side and the shaft may extend in the passageway of the second side beam along a length of the second side beam and have two engagement members in corresponding engagement with the two tabs on the second side of the transparent shield.

The face shield protection device may further comprise an inlet provided through the frame and including a connection for a vacuum source, wherein the inlet is in fluid communication with at least one nozzle extending beneath a bottom surface of the transparent shield, and suction provided through the at least one nozzle pulls atmosphere from beneath the transparent shield creating negative pressure beneath the transparent shield and pulling fresh atmosphere from around the transparent shield into the area beneath the transparent shield. The distal beam of the frame may be tubular and defines a passageway and the at least one nozzle may be contained within the passageway of the distal beam of the transparent shield and may be exposed to the atmosphere beneath the bottom surface of the transparent shield through one or more openings in the distal beam of the frame.

The suspension arm may be adapted for attachment to a stationary object. The connection of the shield assembly to the suspension arm, the connection of the suspension arm to the stationary object, and/or the suspension arm may include one or more hinges and/or pivots, and the suspension arm may comprise more than one segment connected by a hinge and/or pivot. Via the one or more hinges and/or pivots, the shield assembly may be rotatable about an axis extending in a longitudinal direction of the shield assembly, thereby allowing the shield assembly to be rotated upwardly from a position in which a bottom surface of the transparent shield is in an initial substantially horizontal position and is substantially parallel to a horizontal reference plane, to a second position in which the bottom surface of the transparent shield is in a substantially vertical position at an angle to the reference plane. Via the one or more hinges and/or pivots, the shield assembly may be rotatable about an axis extending perpendicular to a top surface and a bottom surface of the transparent shield, thereby allowing the shield assembly to be laterally rotated from a position in which a bottom surface of the transparent shield is in an initial position above and substantially parallel to a first area of a horizontal reference plane, to a second position in which the bottom surface of the transparent shield is above and substantially parallel to a second, different area of the horizontal reference plane. Via the one or more hinges and/or pivots, the shield assembly may be rotatable about an axis extending in a transverse direction of the shield assembly, thereby allowing the shield assembly to be rotated such that an angle of a longitudinal axis of the shield assembly to a horizontal reference plane is changed. The connection of the shield assembly to the suspension arm, the connection of the suspension arm to the stationary object, and/or the suspension arm itself may also be configured to allow a distance between the shield assembly and a horizontal reference plane to be adjusted.

### BRIEF DESCRIPTION OF THE DRAWINGS

FIG. 1 is a front perspective view of the inventive face shield protection device.

FIG. 2 is a top view of the shield assembly of the face shield protection device of FIG. 1.

FIG. 3 is a bottom view of the shield assembly of the face shield protection device of FIG. 1.

FIG. 4 is a proximal end view of the shield assembly of the face shield protection device of FIG. 1.

FIG. 5 is a distal end of the shield assembly of the face shield protection device of FIG. 1.

FIG. 6 is a top view of the shield of the face shield protection device of FIG. 1.

FIG. 7 is a top view of the shield assembly of the face shield protection device of FIG. 1 showing the interior of the distal beam of the frame.

FIG. 8 is a top view of the shield assembly of the face shield protection device of FIG. 1 showing the interior of the second side beam of the frame.

FIG. 9 is a front perspective view of the inventive face shield protection device.

FIG. 10 is a front perspective view of the inventive face shield protection device of FIG. 1 in use in a dentist's office.

### DESCRIPTION OF THE INVENTION

As used herein, unless otherwise expressly specified, all numbers such as those expressing values, ranges, amounts or percentages may be read as if prefaced by the word “about”, even if the term does not expressly appear. “Including”, “such as”, “for example” and like terms means “including/such as/for example but not limited to”.

For purposes of the description hereinafter, spatial orientation terms, as used, shall relate to the referenced embodiment as it is oriented in the accompanying drawings, figures, or otherwise described in the following detailed description. However, it is to be understood that the embodiments described hereinafter may assume many alternative variations and configurations. It is also to be understood that the specific components, devices, features, and operational sequences illustrated in the accompanying drawings, figures, or otherwise described herein are simply exemplary and should not be considered as limiting.

The present invention is directed to a face shield protection device 10 that includes a shield assembly 12 and a suspension arm 14. The shield assembly 12 comprises a frame 16 defining an opening 18 and a transparent shield 20 removably attached to the frame 16 and substantially covering the opening 18. The transparent shield 20 is attached to a first side 22 of the frame 16 in a first receiving engagement in which a portion of the transparent shield 20 is received within a portion of the frame 16 and is attached to an opposite second side 24 of the frame 16 in a second receiving engagement in which a portion of the transparent shield 20 is received within an opposing second side portion of the frame 16, and one or both of the first receiving engagement and the second receiving engagement locks the transparent shield 20 with a portion of the frame 16.

The frame 16 has a proximal beam 26, a distal beam 28, a first side beam 30 extending from a first end 34 of the proximal beam 26 to a first end 36 of the distal beam 28, and a second side beam 32 extending from a second end 38 of the proximal beam 26 to a second end 40 of the distal beam 28. The proximal beam 26, distal beam 28, first side beam 30, and second side beam 32 define an opening 18, which may be substantially rectangular. Each of the proximal beam 26, distal beam 28, first side beam 30, and/or second side beam 32 may be tubular and define an interior passageway. If the proximal beam 26, distal beam 28, first side beam 30, and/or second side beam 32 are tubular, each may have a suitable cross-sectional shape, for example, square, rectangular, or round. The frame 16 may be made of any suitable lightweight material including, for example, plastic or aluminum.

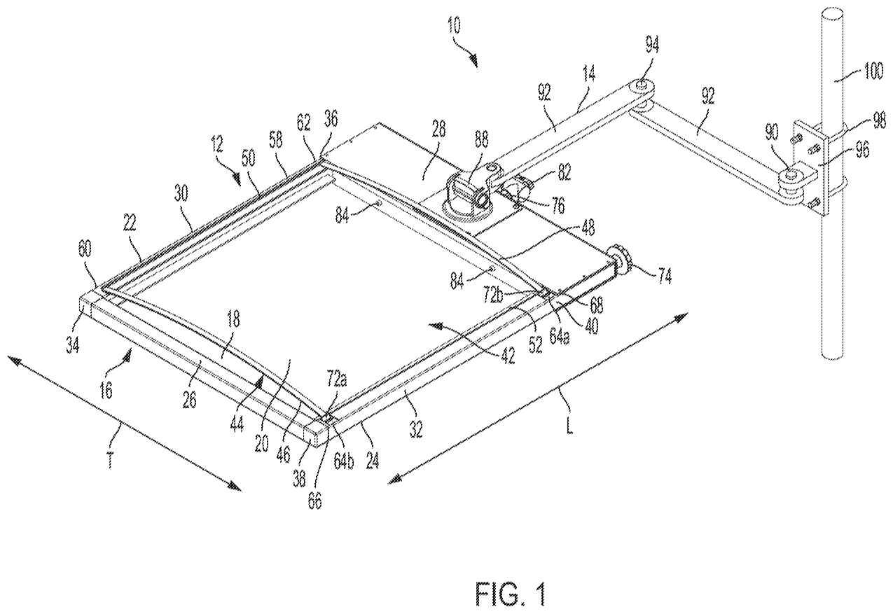

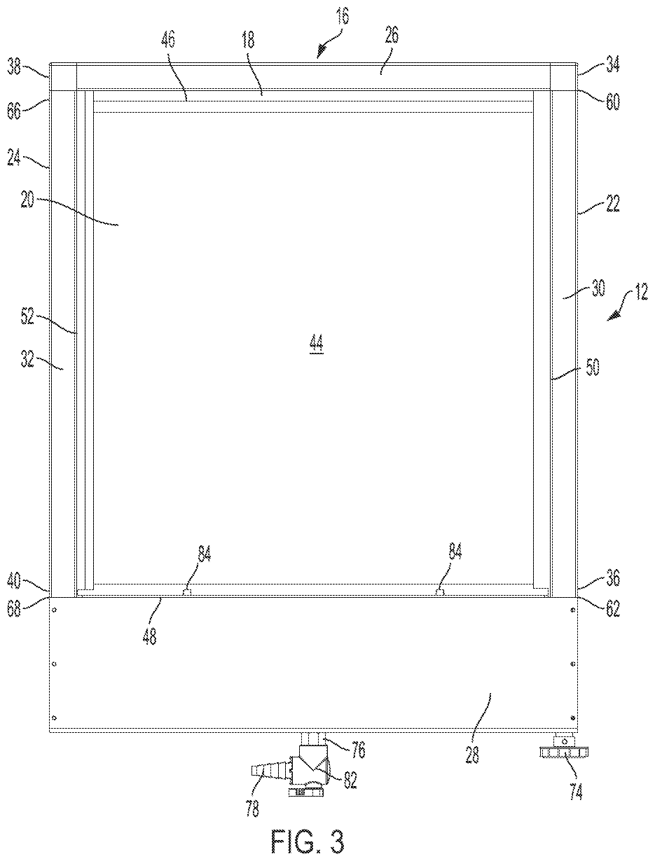

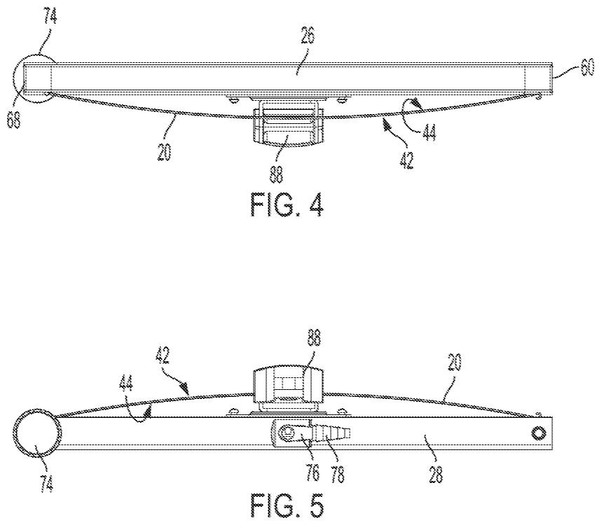

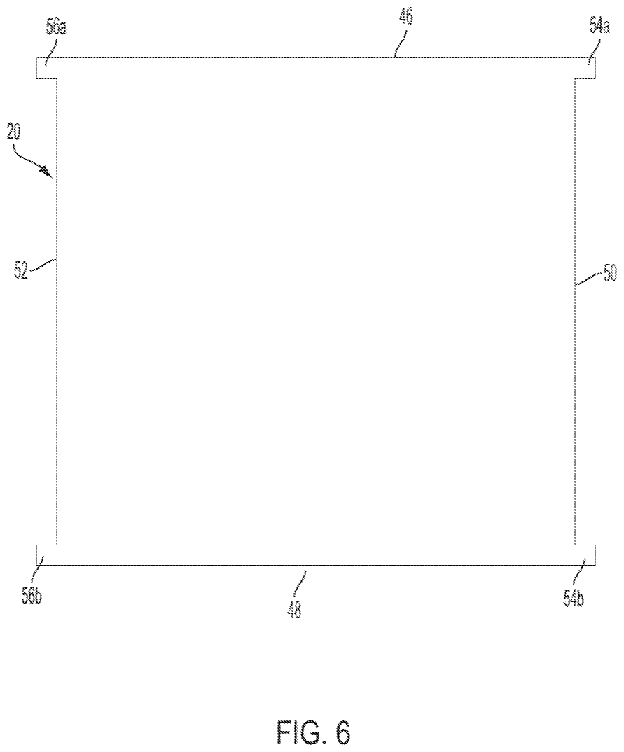

The transparent shield 20 is a thin, flexible sheet and has a shape that corresponds to the shape of the opening 18 defined by the frame 16, such that, when the transparent shield 20 is engaged with the frame 16, the transparent shield 20 covers the opening 18. The transparent shield 20 has a top surface 42 and a bottom surface 44 and a proximal end 46, a distal end 48, a first side 50, and a second side 52 which correspond to the proximal beam 26, distal beam 28, first side beam 30, and second side beam 32, respectively, of the frame 16. At least one tab 54a, 54b extends from the first side 50 and/or at least one tab 56a, 56b extends from the second side 52 of the transparent shield 20. For example, a single tab may extend from a middle area of the first side 50 of the transparent shield 20 and/or the second side 52 of the transparent shield 20 or two tabs 54a, 54b may extend from the first side 50 of the transparent shield 20 and/or two tabs 56a, 56b may extend from the second side 52 of the transparent shield 20 with one tab 54a, 56a adjacent the proximal end 46 of the transparent shield 20 and one tab 54b, 56b adjacent the distal end 48 of the transparent shield 20. The tabs 54a, 54b, 56a, 56b may have a length that is shorter than a length of the first side 50, or the second side 52 of the transparent shield 20 may have different lengths, or if a single tab is present it may extend for the entire length of the first side 50 or the second side 52 of the transparent shield 20. In the embodiment shown in FIGS. 1-8, two tabs 54a, 54b are provided on the first side 50 of the transparent shield 20, each having a length that is less than the length of the first side 50 of the transparent shield 20. Two tabs 56a, 56b are provided on the second side 52 of the transparent shield 20, each having a length that is less than the length of the second side 52 of the shield 50. In each case, one tab 54a, 56a extends adjacent the proximal end 46 of the transparent shield 20 and one tab 54b, 56b extends adjacent the distal end 48 of the shield 50.

The tab or tabs 54a, 54b on the first side 50 of the transparent shield 20 are received by the first side beam 30 of the frame 16, and the tab or tabs 56a, 56b on the second side 52 of the transparent shield 20 are received by the second side beam 32 of the frame 16. The tab or tabs 54a, 54b on the first side 50 of the transparent shield 20 and/or the tab or tabs 56a, 56b on the second side 52 of the transparent shield 20 may be received and locked within the first side beam 30 of the frame 16 and/or the second side beam 32 of the frame 16.

The first side beam 30 may be tubular and have at least one groove 58 extending through the sidewall for receiving at least one tab 54a, 54b extending from the first side 50 of the transparent shield 20 in the passageway defined by the tubular first side beam 30. A single groove 58 may extend along the length of the first side beam 30 from the proximal end 60 of the first side beam 30 to the distal end 62 of the first side beam 30 and may receive two tabs 54a, 54b provided on the first side 50 of the transparent shield 20. Alternatively, the first side 50 of the transparent shield 20 may be provided with a single tab and/or the first side beam 30 may be provided with a series of grooves each corresponding to one of the tabs extending from the first side 50 of the transparent shield 20. In a further alternative, the first side beam 30 may include at least one protrusion having a groove. A tab having a length the same as or different from the length of the groove may be provided on the first side 50 of the transparent shield 20 and received within the groove.

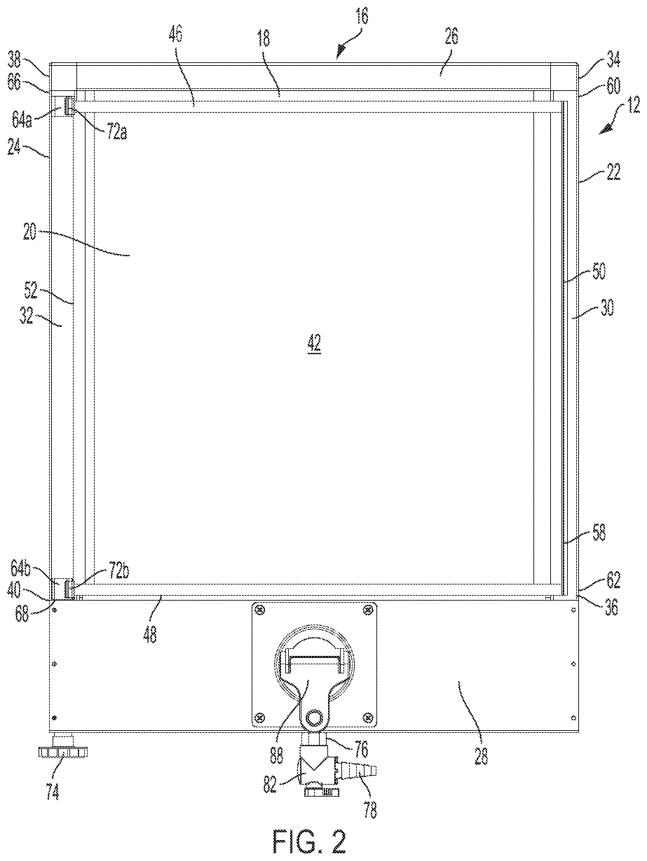

The second side beam 32 may be tubular and have at least one opening 64a, 64b through the sidewall for receiving at least one tab 56a, 56b extending from the second side 52 of the transparent shield 20. As shown in FIG. 2, the second side beam 32 has two openings 64a, 64b, one opening 64a adjacent the proximal beam 26 and one opening 64b adjacent the distal beam 28. The transparent shield 20 has two tabs 56a, 56b extending from the second side 52 of the shield 20, each tab 56a, 56b corresponding to one of the openings 64a, 64b. Alternatively, the second side 52 of the transparent shield 20 may be provided with a single tab, and the second side beam 32 may be provided with a single opening. The single opening may extend from the proximal end 66 of the second side beam 32 to the distal end 68 of the second side beam 32.

A shaft 70 is contained within the passageway of the second side beam 32. At least one engagement member 72a, 72b extends from the shaft 70. The engagement member 72 may be a protrusion extending from the shaft 70 through the opening 64a, 64b. When the tab 56a, 56b on the second side 52 of the transparent shield 20 passes into the opening 64a, 64b in the second side beam 52, the engagement member 72a, 72b is configured to, upon rotation of the shaft 70, engage the tab 56a, 56b between the protrusion and the side of the opening 64a, 64b in the second side member 32 to lock the tab 56a, 56b within the passageway of the second side member 32. Alternatively, the engagement member 72a, 72b may be a disc having a toothed or knurled surface, for example, a gear or a knurled wheel, that frictionally engages the tab 56a, 56b upon rotation of the shaft 70.

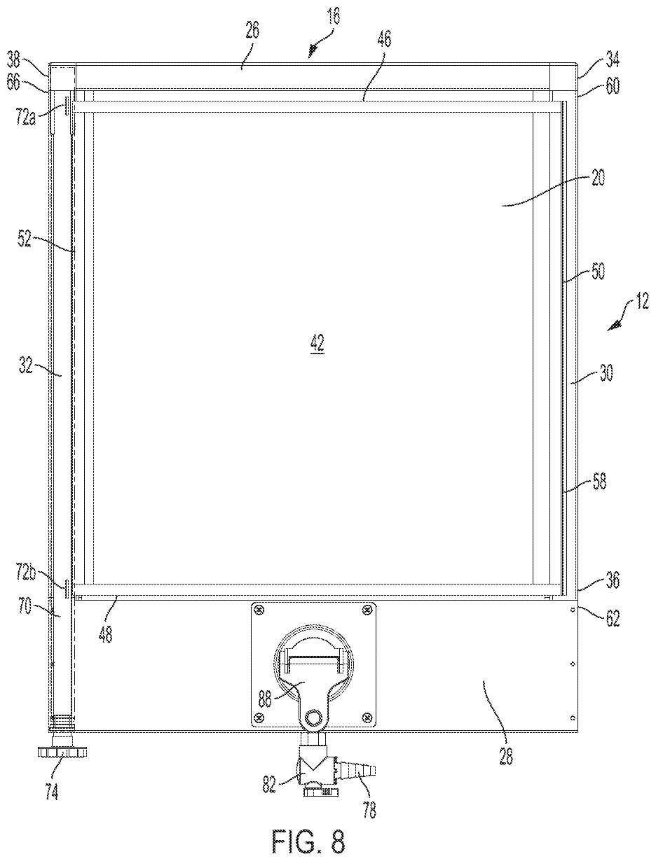

As shown in FIG. 8, the shaft 70 may extend in the passageway of the second side beam 32 along the entire length of the second side beam 32 and have two engagement members 72a, 72b, one adjacent the proximal beam 26 and one adjacent the distal beam 28, in corresponding engagement with two tabs 56a, 56b on the second side 52 of the transparent shield 20.

A handle 74 connected to the shaft 70 is provided on the exterior of the frame 16. As shown in FIGS. 1-5, the handle 74 may be a circular knob and may be connected to a distal end of the shaft 70.

In order to attach the transparent shield 20 to the frame 16, the tabs 54a, 54b on the first side 50 of the transparent shield 20 are inserted into the groove 58 in the first side member 30 and the tabs 56a, 56b on the second side 52 of the transparent shield 20 are inserted into the openings 64a, 64b in the second side member 32. The shaft 70 is rotated within the passageway of the second side beam 32 and each of the tabs 56a, 56b on the second side 52 of the transparent shield 20 are locked between the engagement member 72a, 72b and the side of the opening 64a, 64b in the second side beam 32. Alternatively, if the engagement members are a gear or a knurled wheel, the engagement members pull the tabs 56a, 56b into the passageway of the second side beam 32 and lock the tabs 56a, 56b within the passageway of the second side beam 32. To remove the transparent shield 20, the process is reversed by rotating the shaft 70 in the opposite direction such that the tabs 56a, 56b are released from engagement with the engagement members 72a, 72b and can be removed from the openings 64a, 64b in the second side beam 32. The tabs 54a, 54b on the first side 50 of the transparent shield 20 can then be removed from the groove 58 in the first side beam 30.

In another embodiment, the first side beam 30 may also be configured to provide a locking engagement with the transparent shield 20 by providing at least one opening in the first side beam 30, a shaft within the first side beam 30, and at least one engagement member attached to the shaft.

As discussed above, while the illustrated embodiment has two tabs 56a, 56b on the second side 52 of the transparent shield 20, two openings 64a, 64b in the second side beam 32, and two engagement members 72a, 72b, the face shield device may include any number of corresponding tabs on the shield, openings in the second side beam, and engagement members. Further, the face shield device may include different numbers of tabs, openings, and engagement members. For example, two tabs, a single opening, and two engagement members; one tab, one opening, and one engagement member; one tab, one opening, and more than one engagement member; one tab, more than one opening, and more than one engagement member; or more than one tab, more than one opening, and one engagement member.

Similarly, while the illustrated embodiment has two tabs 54a, 54b on the first side 50 of the transparent shield 20 and one groove 58 in the first side beam 30, the face shield device may include any number of corresponding tabs on the first side of the shield and grooves in the first side beam. Further, the face shield device may include different numbers of tabs and grooves. For example, one tab and one groove; three tabs and one groove; or three tabs and three grooves.

The top surface 42 of the transparent shield 20 may have a convex curvature in the transverse direction and/or a convex curvature in the longitudinal direction. Curvature in the transverse direction may be created by providing a transparent shield 20 where the length of the proximal end 46 and the distal end 48 is greater than a length of the proximal beam 26 and distal beam 28 of the frame 16, such that installation of the transparent shield 20 in the frame 16 causes the transparent shield 20 to arch. The transparent shield 20 may be made out of plastic, plexiglass, flexible clear polycarbonate, polyester plastic, or any other suitable material.

An inlet 76 extending thorough the frame 16 and including a connection 78 for a vacuum source, for example, a vacuum hose in a dental and/or medical office. The connection 78 may be provided on an exterior of the frame 16. The inlet 76 is in fluid communication with tubing 80 provided adjacent or within the distal beam 28 of the frame 16. The connection 78 may take any form suitable for attaching a suction hose to the inlet, for example, a barbed fitting or a quick release fitting. The connection 78 may include a valve 82 that shuts off and/or adjusts the flow of the suction through the inlet 76.

The tubing 80 that is in fluid communication with the inlet 76 is also in fluid communication with at least one nozzle 84 located beneath the bottom surface 44 of the transparent shield 20. Alternatively, the nozzle 84 may be directly connected to the inlet 76.

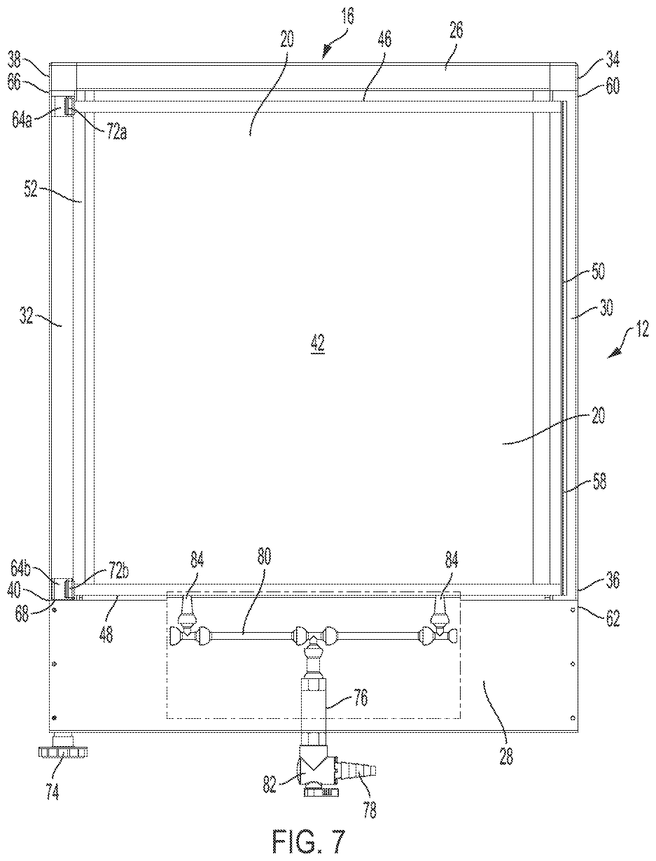

As shown in FIG. 7, the face shield protection device may include more than one nozzle 84 arranged along and extending from a central tube 80 connected to the inlet 76. The nozzles 84 may be evenly spaced along the length of the distal beam 28 of the frame 16 or may be placed at different distances from one another. Alternatively, a plurality of inlets 76 could be provided and each of the nozzles 84 could be directly connected to an inlet 76.

The nozzles 84 suction the atmosphere from beneath the transparent shield 20, creating negative pressure beneath the transparent shield 20 and pulling fresh atmosphere from around the transparent shield 20 into the area beneath the transparent shield 20. The nozzles 84 may take any suitable shape including conical and fan shaped.

The distal beam 28 of the frame 16 may be tubular and the tubing 80 and/or nozzles 84 may be contained within the passageway defined by the distal beam 80 of the frame 16. The openings of the nozzles 84 may be exposed to the atmosphere beneath the bottom surface 44 of the transparent shield 20 through one or more openings in the distal beam 28 of the frame 16.

Alternatively, instead of the nozzles, a hose or tube may be provided entirely or partially around the outside periphery of the transparent shield 20. For example, the hose may extend around 95% of the periphery of the transparent shield 20. The hose may include several perforations or openings spaced apart, in a regular pattern or a random pattern, along the length of the hose. The perforations or openings may be large enough to generate enough suction (negative pressure) to remove any aerosol in the area beneath the bottom surface 44 of the transparent shield 20. The perforations or openings may extend completely along the entire length of the hose or may only be present in certain areas of the hose. The hose may be permanently or detachably attached to the shield.

A suspension arm 14 is attached to the shield assembly 12 and used to connect the shield assembly 12 to a stationary object, for example, a wall, a ceiling, a pole, or a chair. A connector 88 to which the suspension arm 14 is attached may be provided on the frame 16 of the shield assembly 12.

The connection 88 provided for attachment of the shield assembly 12 to the suspension arm 14 and/or the connection 90 of the suspension arm 14 to the stationary object may include a hinge and/or pivot. The suspension arm 14 itself may also be adjustable. For example, the suspension arm 14 may comprise more than one segment 92 attached by a connection 94 that may include a hinge and/or pivot, thereby allowing the suspension arm 14 to be folded into a compact structure where the shield assembly 12 is close to the stationary object and extended to suspend the shield assembly 12 at a distance away from the stationary object.

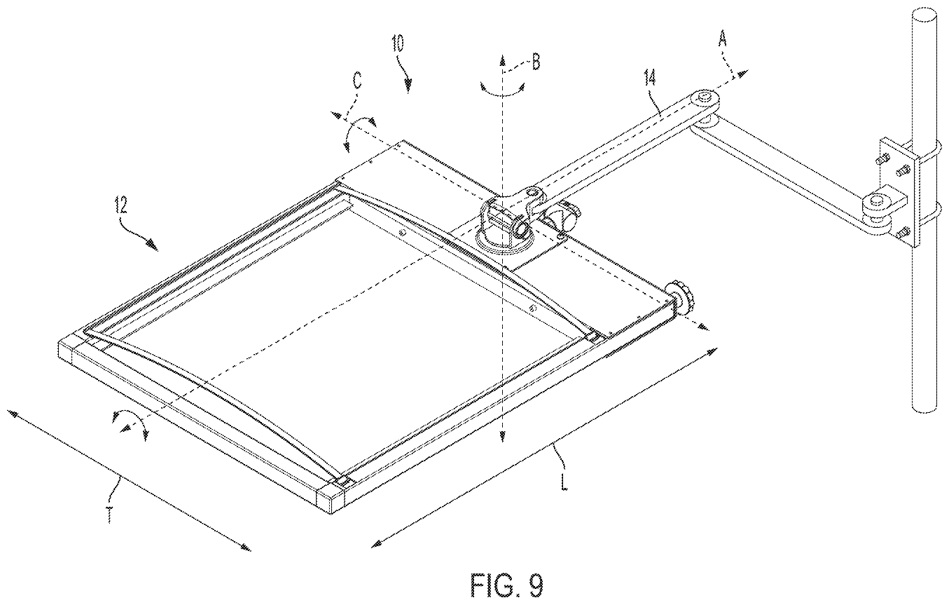

As shown in FIG. 9, via the connection 88 provided for attachment of the shield assembly 12 to the suspension arm 14, the connection 90 provided for the attachment of the suspension arm 14 to the stationary object, and or the connection 94 provided for attachment of segments 92 of the suspension arm 14, the shield assembly 12 may be rotated around a first axis A extending in the longitudinal direction L of the shield assembly 12, thereby allowing the shield assembly 12 to be rotated upwardly from a position in which the bottom surface 44 of the transparent shield 20 is in an initial substantially horizontal position and is substantially parallel to a horizontal reference plane, for example, the floor, to a second position in which the bottom surface 44 of the transparent shield 20 is in a substantially vertical position at an angle to the reference plane. The shield assembly 12 may be rotated upwardly via the connection 88 provided for attachment of the shield assembly 12 to the suspension arm 14, the connection 90 provided for the attachment of the suspension arm 14 to the stationary object, and or the connection 94 provided for attachment of segments 92 of the suspension arm 14 through an angle of at least 20°, for example, at least 45°, and/or through an angle of at most 180°, for example, at most 90°. Overall, the shield assembly 12 may be rotated upwardly via the connection 88 provided for attachment of the transparent shield 20 assembly to the suspension arm 14 and/or the connection 90 provided for the attachment of the suspension arm 14 to the stationary object through an angle of 20°-180°, for example, 45°-180°, 20°-90°, or 90°-180°.

As shown in FIG. 9, via the connection 88 provided for attachment of the shield assembly 12 to the suspension arm 14, the connection 90 provided for the attachment of the suspension arm 14 to the stationary object, and or the connection 94 provided for attachment of segments 92 of the suspension arm 14, the shield assembly 12 may be rotated about a second axis B extending perpendicular to the top surface 42 and bottom surface 44 of the shield 20, thereby allowing the shield assembly 12 to be laterally rotated (swiveled) from a position in which the bottom surface 44 of the transparent shield 20 is in an initial position above and substantially parallel to a first area of the horizontal reference plane, to a second position in which the bottom surface 44 of the transparent shield 20 is above a second, different area of the horizontal reference plane, yet is still substantially parallel to the horizontal reference plane. The shield assembly 12 may be rotated laterally upwardly via the connection 88 provided for attachment of the shield assembly 12 to the suspension arm 14, the connection 90 provided for the attachment of the suspension arm 14 to the stationary object, and or the connection 94 provided for attachment of segments 92 of the suspension arm 14 through an angle of at least 45°, for example, at least 90°, and/or through an angle of at most 180°, for example, at most 90°. Overall, the shield assembly may be rotated through an angle of 45°-180°, for example, 90°-180° or 45°-90°.

As shown in FIG. 9, via the connection 88 provided for attachment of the shield assembly 12 to the suspension arm 14, the connection 90 provided for the attachment of the suspension arm 14 to the stationary object, and or the connection 94 provided for attachment of segments 92 of the suspension arm 14, the shield assembly 12 may be rotated about a third axis C extending in the transverse direction T of the shield assembly 12, thereby allowing the shield assembly 12 to be rotated such that the angle of the longitudinal axis L of the shield assembly 12 to the horizontal reference plane is changed. The shield assembly 12 may be rotated from a position in which the angle of the longitudinal axis L of the shield assembly 12 relative to the horizontal reference plane is 0° to a position in which the angle of the longitudinal axis of the shield assembly relative to the horizontal reference plane is 60°. The angle of the longitudinal axis of the shield assembly relative to the horizontal reference plane may be 0° or at least 10°, for example, at least 20° or at least 30°, and/or may be at most 60°, for example, at most 50° or at most 45°. Overall, the shield assembly may be rotated via the connection through an angle of 0°-60°, for example, 0°-50°, 0°-45°, 10°-60°, or 10°-45°.

The connection 88 provided for attachment of the shield assembly 12 to the suspension arm 14, the connection 90 provided for the attachment of the suspension arm 14 to the stationary object, and or the connection 94 provided for attachment of segments 92 of the suspension arm 14 may also allow the distance (height) between the shield assembly 12 and the reference plane to be adjusted.

The connection 90 of the suspension arm 14 to the stationary object may include a plate 96 to which the suspension arm 14 is connected and at least one U-shaped bolt 98 for attaching the suspension arm 14 to a pole 100.

Alternatively, the face shield assembly 12 may be connected to a stand comprising a vertical post. The shield assembly may be attached to a first end of the vertical post while a second end of the vertical post is attached to a base. The base may be a solid base or may comprise a plurality of legs. The height of the vertical post may be adjustable. For example, the vertical post may comprise two pieces with the first piece inserted into the second piece. The first piece may include a retractable protrusion and the second piece may include a plurality of holes. By engaging the retractable protrusion with one of the plurality of holes, the overall height of the vertical post may be set. By retracting the retractable protrusion and moving the first piece relative to the second piece such that the retractable protrusion engages another of the plurality of holes, the height of the vertical post may be changed. Other suitable methods may be provided for allowing the height of the vertical post to be adjusted, for example, a hydraulic or pneumatic piston may be used for moving and holding the first piece relative to the second piece.

Alternatively, the suspension arm 14 may be a rigid and adjustable hose such that it may be connected to the inlet 76 and provide the vacuum source to the shield assembly 12.

A handle may be attached to the shield assembly to assist the user in positioning the shield assembly during use.

An air moving device may optionally be connected adjacent the proximal beam 26 of the frame 16 opposite the nozzles 84. The air moving device may be a fan or another suitable device that causes the air in the area beneath the bottom surface 44 of the shield assembly 12 to be circulated. The air moving device may be rechargeable or battery powered and may be permanently or detachably attached to the shield assembly 12. The air flow provided by the air moving device helps to alleviate fog build up and aerosol build up on the transparent shield 20 and directs aerosol toward the nozzles 84, thereby facilitating the removal of the aerosol by the suction source.

Any or all of the parts of the face shield protection device 10 including the frame 16, the shield 12, and the suspension arm 14 may be made from autoclaveable materials so that they may be sterilized and reused in a dental or medical setting.

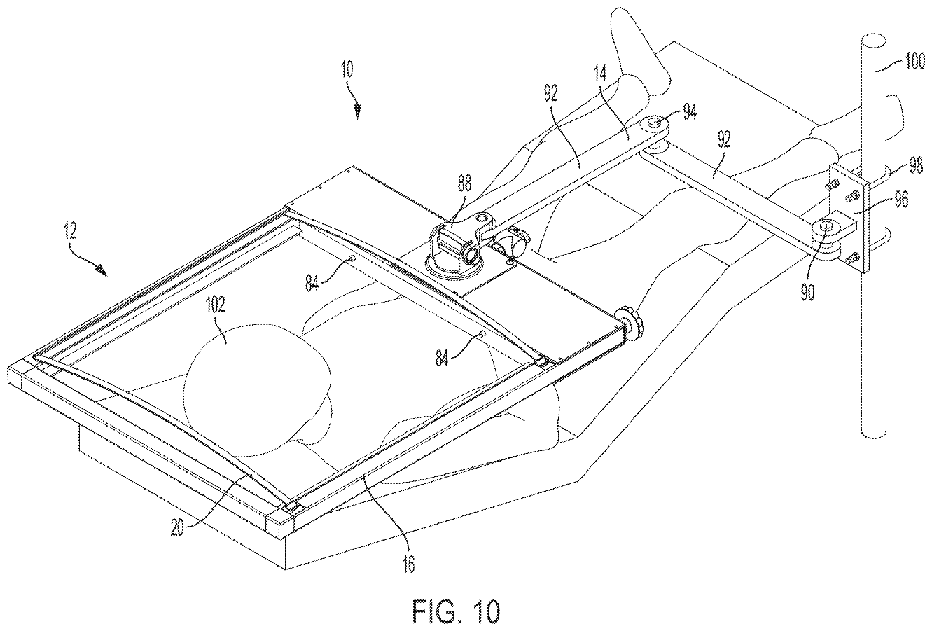

In use, as shown in FIG. 10, the shield assembly 12 of the face shield protection device 10 hovers above or in front of a person's face 102, utilizes suction to eliminate aerosol, and may optionally utilize air flow to reduce fogging, reduce settling of aerosol on the transparent shield 20, and aid suctioning. The face shield protection device 10 may be used in a variety of settings and particularly may be placed above or in front of a patient's face 102 during a dental or medical procedure, especially a procedure where a dental or medical professional, for example, a dentist or an ear, nose, and throat (ENT) doctor or technician, needs to access the patient's oral cavity, nose, or face.

The transparent shield 20 covers a person's face 102 and has a length sufficient to extend beyond both the person's chin and nose. The transparent shield 20 may curve around at least partially around the side of the person's face 102. In the longitudinal direction L, the shield assembly 12 may have a length sufficient to extend beyond both the person's chin and at least part of the person's forehead, and in a transverse direction, the shield assembly 12 may have a width sufficient to cover the person's face from side to side.

The adjustability of the connection 88 of the shield assembly 12 to the suspension arm 14, the connection 90 of the suspension arm 14 to the stationary object, and/or the adjustability of the suspension arm 14 itself allows the shield assembly 12 to be placed in a position that sufficiently covers the person's face 102 yet allows access to the person's face 102 beneath the shield 12. For example, the shield assembly 12 may be placed in a position that sufficiently covers a dental patient's face 102 yet allows a dentist or dental technician to access the patient's mouth beneath the transparent shield 20.

Rotation of the shield assembly 12 about the first axis A allows the shield assembly 12 to be rotated from an initial substantially horizontal position above the face 102 of the person and substantially parallel to the floor, to a second position in which the shield assembly 12 is in a substantially vertical position at the side of the person's face 102 and is at an angle to the floor. This movement allows the shield assembly 12 to be easily positioned to the side to allow, for example, a dental patient to enter a dental chair and then be flipped in a downward direction such that the shield assembly 12 is suspended above the patient's face 102.

Rotation of the shield assembly 12 about the second axis B allows the shield assembly 12 to be laterally rotated (swiveled) from a position in which the shield assembly 12 is in an initial position above the face 102 of the patient and is substantially parallel to the patient's face 102 and/or the floor, to a second position in which the shield assembly 12 is no longer over the person's face 102, but is still in a plane that is substantially parallel to the patient's face 102 and/or the floor.

Rotation of the shield assembly about the third axis C allows the shield to be placed parallel to the person's face 102 when the person is in a reclined position that is not completely horizontal or flat.

Whereas particular aspects of this invention have been described above for purposes of illustration, it will be evident to those skilled in the art that numerous variations of the details of the present invention may be made without departing from the invention.

The invention claimed is:

1. A face shield protection device comprising:
a shield assembly comprising a frame defining an opening and a transparent shield removably attached to the frame and substantially covering the opening; and
a suspension arm attached to the shield assembly,
wherein the transparent shield is attached to a first side of the frame in a first receiving engagement in which a portion of the transparent shield is received within a portion of the frame and is attached to an opposite second side of the frame in a second receiving engagement in which a portion of the transparent shield is received within an opposing second side portion of the frame,
and one or both of the first receiving engagement and the second receiving engagement locks the transparent shield with a portion of the frame.

2. The face shield protection device of claim 1, wherein the frame comprises a proximal beam, a distal beam, a first side beam extending from a first end of the proximal beam to a first end of the distal beam, and a second side beam extending from a second end of the proximal beam to a second end of the distal beam, wherein the proximal beam, the distal beam, the first side beam, and the second side beam define the opening.

3. The face shield protection device of claim 2, wherein the proximal beam, the distal beam, the first side beam, and/or second side beam are tubular and define a passageway.

4. The face shield protection device of claim 2, wherein the transparent shield has a proximal end, a distal end, a first side, and a second side which correspond to the proximal beam, the distal beam, the first side beam, and the second side beam, respectively, of the frame.

5. The face shield protection device of claim 4, wherein the first side beam and the second side beam are tubular, each defining a passageway, at least one tab extends from the first side of the transparent shield and at least one tab extends from the second side of the transparent shield, and the at least one tab on the first side of the transparent shield is received by a groove in the first side beam of the frame in the first receiving engagement, and the at least one tab on the second side of the transparent shield is received in an opening through a sidewall of the second side beam of the frame in the second receiving engagement.

6. The face shield protection device of claim 5, wherein the at least one tab on the first side of the transparent shield is locked within the first side beam frame and/or the at least one tab on the second side of the transparent shield is locked within the second side beam of the frame.

7. The face shield protection device of claim 5, wherein a shaft is contained within the passageway of the second side beam and at least one engagement member extends from the shaft and when the at least one tab on the second side of the transparent shield passes into the opening in the second side beam, the engagement member is configured to, upon rotation of the shaft, engage the at least one tab to lock the at least one tab within the passageway of the second side member.

8. The face shield protection device of claim 7, wherein the engagement member is a protrusion extending from the shaft through the opening, and the at least one tab is locked between the protrusion and a side of the opening in the second side member.

9. The face shield protection device of claim 5, wherein the transparent shield comprises two tabs extending from opposite ends of the second side and the shaft extends in the passageway of the second side beam along a length of the second side beam and has two engagement members in corresponding engagement with the two tabs on the second side of the transparent shield.

10. The face shield protection device of claim 1, further comprising an inlet provided through the frame and including a connection for a vacuum source,
wherein the inlet is in fluid communication with at least one nozzle extending beneath a bottom surface of the transparent shield, and suction provided through the at least one nozzle pulls atmosphere from beneath the transparent shield creating negative pressure beneath the transparent shield and pulling fresh atmosphere from around the transparent shield into the area beneath the transparent shield.

11. The face shield protection device of claim 10,
wherein the frame comprises a proximal beam, a distal beam, a first side beam extending from a first end of the proximal beam to a first end of the distal beam, and a second side beam extending from a second end of the proximal beam to a second end of the distal beam, and the proximal beam, the distal beam, the first side beam, and the second side beam define the opening, and
wherein the distal beam of the frame is tubular and defines a passageway and the at least one nozzle is contained within the passageway of the distal beam and is exposed to the atmosphere beneath the bottom surface of the transparent shield through one or more openings in the distal beam of the frame.

12. The face shield protection device of claim 1, wherein the suspension arm is adapted for attachment to a stationary object.

13. The face shield protection device of claim 12, wherein the connection of the shield assembly to the suspension arm, the connection of the suspension arm to the stationary object, and/or the suspension arm includes one or more hinges and/or pivots.

14. The face shield protection device of claim 13, wherein the suspension arm comprises more than one segment connected by a hinge and/or pivot.

15. The face shield protection device of claim 13, wherein, via one or more of the hinges and/or pivots, the shield assembly is rotatable about an axis extending in a longitudinal direction of the shield assembly, thereby allowing the shield assembly to be rotated upwardly from a position in which a bottom surface of the transparent shield is in an initial substantially horizontal position and is substantially parallel to a horizontal reference plane, to a second position in which the bottom surface of the transparent shield is in a substantially vertical position at an angle to the reference plane.

16. The face shield protection device claim 13, wherein, via one or more of the hinges and/or pivots, the shield assembly is rotatable about an axis extending perpendicular to a top surface and a bottom surface of the transparent shield, thereby allowing the shield assembly to be laterally rotated from a position in which a bottom surface of the transparent shield is in an initial position above and substantially parallel to a first area of a horizontal reference plane, to a second position in which the bottom surface of the transparent shield is above and substantially parallel to a second, different area of the horizontal reference plane.

17. The face shield protection device of claim 13, wherein, via the one or more hinges and/or pivots, the shield assembly is rotatable about an axis extending in a transverse direction of the shield assembly, thereby allowing the shield assembly to be rotated such that an angle of a longitudinal axis of the shield assembly to a horizontal reference plane is changed.

18. The face shield protection device of claim 13, wherein the connection of the shield assembly to the suspension arm, the connection of the suspension arm to the stationary object, and/or the suspension arm itself are configured to allow a distance between the shield assembly and a horizontal reference plane to be adjusted.

19. The face shield protection device of claim 1, wherein a top surface of the transparent shield has a convex curvature in the transverse direction and/or a convex curvature in the longitudinal direction.

20. The face shield protection device of claim 1, wherein the transparent shield is a flexible sheet and has a shape that corresponds to the shape of the opening defined by the frame.
